(19) United States
(12) Patent Application Publication
(10) Pub. No.: US 2023/0251176 A1
(43) Pub. Date: Aug. 10, 2023

(54) DEVICE AND METHOD FOR DETECTING THE FLOCCULATION THRESHOLD OF A COLLOIDAL MEDIUM, IN PARTICULAR A MEDIUM COMPRISING ASPHALTENES, BY THE ADDITION OF ALIPHATIC SOLVENT

(71) Applicant: TOTAL RAFFINAGE CHIMIE, COURBEVOIE (FR)

(72) Inventors: Ahmad AL FARRA, LE HAVRE (FR); Frédéric JOSE, BARRAUTE CAMU (FR); Sandra FANTOU, LE HAVRE (FR); Jérôme OLIVIER, LE HAVRE (FR)

(73) Assignee: TotalEnergies OneTech, COURBEVOIE (FR)

(21) Appl. No.: 18/011,957

(22) PCT Filed: Jun. 21, 2021
(86) PCT No.: PCT/EP2021/066880

§ 371 Date: Dec. 21, 2022

(30) Foreign Application Priority Data
Jun. 23, 2020 (EP) 20305689.0

(51) Int. Cl. G01N 15/00; G01N 31/02; G01N 1/38

(52) U.S. Cl. CPC G01N 15/00; G01N 1/38; G01N 31/02; G01N 2015/0092

(57) ABSTRACT

A device for measuring the flocculation threshold of a colloidal medium, and a method for measuring the flocculation threshold of a colloidal medium by the addition of aliphatic solvent using the device, including the step of determining the flocculation after the addition of the amount of aliphatic solvent necessary for flocculation.

### TECHNICAL FIELD

The subject of the invention is a new method for detecting the flocculation of asphaltenes, as well as an associated detection device for measuring light in heavy hydrocarbon products, waste oils, dirty water or any product containing an emulsion and, in particular, for measuring the flocculation threshold of a colloidal medium.

### PRIOR ART

Petroleum products, and in particular fuel oils or petroleum distillation residues, generally referred to as “black products” in the industry, are colloidal systems consisting of asphaltenes - namely heavy, highly aromatic molecules with paraffinic side-chains - which are dispersed (or also called “peptised”) in the form of micelles in an oily phase. These colloidal systems may be destabilised more or less easily, for example by thermal cracking or by dilution. Thus, in a refinery, the conversion process, known as visbreaking, may lead to the precipitation of asphaltenes under the effect of high process temperatures (generally above 400° C.). Similarly, the constitution of mixtures containing such colloidal systems may generate precipitation of these asphaltenes by flocculation, particularly if the dilution environment is of the paraffinic type.

It is therefore necessary to know or estimate the characteristics of these asphaltenes in black products, such as a petroleum product or a mixture of hydrocarbon products, in order to assess their intrinsic stability, as well as their associated stability reserve. Indeed, the higher the stability reserve, the less the black product will be subject to problems of asphaltene precipitation, or compatibility by dilution with other chemical species, in particular paraffinic bases.

It should be noted that fuel oils or petroleum residues consist of a maltenic matrix (resins + paraffins) and asphaltenes dispersed in colloidal form. Asphaltenes which have very aromatic characteristics are insoluble with paraffins which have aliphatic characteristics. For a residue to be stable, it is necessary for the asphaltenes to be kept in suspension (or dispersed or peptised) in the oil matrix. The peptisation of asphaltenes is ensured by resins which have both aromatic characteristics and aliphatic characteristics. When a residue has been destabilised, the asphaltenes flocculate by agglomerating in the form of large particles which may cause clogging of filters in the various treatment units, or even to the deterioration of the metallurgy, for example fouling the pipes which leads to a loss of energy efficiency and pipe capacity.

The characteristic known as S-value, or even intrinsic stability, for example of a black product, is defined in the industry and in the ASTM D7157-18 standard (Revision 2018) by the following expression:

S=aromaticity of maltenes/aromaticity of asphaltenes, i.e. S=So/(1-Sa), wherein,
So represents the ability of the medium to solubilise the asphaltenes, namely the aromatic characteristics of the medium. The more aromatic it is, the greater the So will be.
Sa is the aromatic characteristics of asphaltenes.
1-Sa represents the aromaticity of the medium necessary to solubilise the asphaltenes present.

If S>1, the asphaltenes are peptised and are therefore stable. S-1 represents the stability reserve (the higher the reserve, the less the black product will be subject to precipitation or compatibility problems).

The severity of a thermal shock, such as that brought about by distillation or visbreaking directly effects the aromaticity of the asphaltenes since the thermal cracking causes the alkyl chains to be cut and the asphaltenes to condense. The more condensed and less branched asphaltenes (weaker Sa) will need a stronger solvent to remain dispersed. Thus, the knowledge of the value of S, linked to that of Sa, will make it possible to specify the settings of the operating conditions of the unit concerned so that it may be operated without the risk of asphaltene precipitation, and consequently, to meet the various quality requirements of the operator.

Furthermore, knowledge of the solvent power values So and the aromatic characteristics of the asphaltenes Sa is necessary to optimise the combination of the various components of the fuel oils. Thus, if a fluxing agent (product capable of lowering the viscosity of a mixture) of low solvent power is added to a black product, for example visbroken, and having high So and low Sa values, the value of the So mixture is reduced, which may lead to a destabilisation of the black product, and consequently to flocculation of the asphaltenes, because the resulting So and Sa values would be too low to satisfy the S>1 relationship, i.e. the condition for said asphaltenes to be peptised, and therefore stable.

Usually, we determine the values of S and Sa in the laboratory, and then by calculation So, of a black product by a step dilution using a paraffinic solvent of said black product, previously mixed with an aromatic solvent. The moment when flocculation occurs is noted. The measurement is repeated for at least one other mixture with a different dilution rate. In this way, results are obtained which make it possible, by linear correlation, to obtain the desired values of S and Sa, then to deduce So by calculation.

Experimentally, the flocculation threshold in a given mixture may be detected using various optical probes operating in the infrared (IR) or near infrared (NIR).

For example, the technique described in patents FR-A-2 596 522 or US-A-4 628 204, from Texaco Belgium SA company, makes it possible to measure by IR the flocculation threshold of a colloidal solution during its dilution. This measurement requires the correct choice of the optical measurement probe (there are various probes) depending on the nature and in particular the presence of asphaltenes to a greater or lesser extent in the black product to be tested. If the operator makes the wrong choice, it is then necessary to clean the equipment, then prepare the sample again for a new measurement with another probe, which results in a loss of time that may exceed one hour of operator time, whereas the time of an analysis is approximately 1.5 to 2 hours, especially if it is the choice of a different probe that proves to be judicious.

Another example is the method developed by Shell company, in collaboration with its Dutch partner Zematra, a manufacturer of analysis equipment. This method, wherein the detection of the flocculation threshold of the colloidal medium is carried out using a single probe, consisting of a simple optical fibre surrounded by glass, is unfortunately not usable for the entire range of black products. Indeed, the systematic heating of the sample to 150° C., in addition to the safety problems, can result in, for certain types of black products, degradations detrimental to the measurement of the flocculation threshold. The time taken for an analysis, is relatively long since it may take more than 5 hours.

Another method is also proposed for measuring the S value on black products with a “Porla” device, manufactured by the Finnish company FMS (Finnish Measurement Systems Ltd), and marketed by the English company Med-Lab. This instrument uses a continuous flow measuring cell for the sample to be analysed with optical detection of the flocculation threshold by means of a prism operating in total reflection. The measurement range is very wide and a result is always available, even with black products whose flocculation threshold is deemed difficult to measure. However, these results are obtained after modifications of the operating parameters of the method, which then become a function of the nature of the product, which is unacceptable when the range of products to be analysed is very variable, as in the oil industry.

The document DE3714755A1 describes a device for measuring the size of flakes in a fluid. The device has a measuring channel located between optical elements that may be moved relative to each other in order to adjust the space between them. The space between the optical elements also forms an angle in order to retain flakes of different sizes along the optical elements. The dimensions of this space are thus chosen according to the dimensions of the flakes to be measured. By measuring the luminosity along the optical elements the dimensions of the flakes held between the two optical elements may be determined. No measurement of the flocculation threshold is described.

The document US2010/053622A1 describes an analysis system for quantify inhomogeneities contained in a fluid sample. This system uses an optical lens, such as a microscope, to focus a light beam on the sample. For this purpose, the sample holder may be moved closer or further from the lens in order to position the sample in a focal plane. The luminous intensity transmitted through the sample is measured by a detector located on the opposite side of the lens to the sample. The sample is moved in a predetermined pattern in the focal plane so that the luminous intensity passing through it is measured along a path representative of the sample. The luminous intensity is strongly attenuated when it encounters a particle, allowing the characterisation and quantification of the particles present in the sample. The movement of the sample thus has the sole function of detecting particles.

The document WO2005003754A2 describes an automatic dosing device for determining the incompatibility of oil products. The detection system consists of a fibre optic light transmission spectrometer, the liquid to be measured passing through a 100 µm thick optical cell which is not detailed. The instrument is equipped with a circuit connected to various chambers and pumps allowing the introduction of a petroleum product, an aliphatic solvent, an aromatic solvent and an auxiliary solvent into a thermostatically controlled mixing container used for the dosage. The sample measurement time is 1 to 2 hours.

There is now a standard (ASTM D7157-18 -Revision 2018) for the determination of S, Sa, So values, which may be implemented by means of a device and a method described in document EP1751518 B1.

Document EP1751518 B1 describes a method for measuring the flocculation threshold wherein at least two light emitting and receiving probes are introduced into the medium to be measured, these probes operate by optical transmission at detection areas of distinct dimensions. It is then determined which of the two probes is suitable for the measurement by determining the transmission threshold of the medium before the addition of aliphatic solvent. Finally, the flocculation is determined with the aid of the probe thus designated after adding the amount of aliphatic solvent necessary for flocculation. In particular, one of the probes operates in indirect transmission by reflection. The method and the device described allow the selection of the most suitable probe for the measurement from various probes introduced into the same medium, in particular after the addition of aliphatic solvent. Thus, the device may be switched from one probe to another after adding solvent. These probe changes, which correspond to changes in the optical path travelled by the light beam between a transmitter and its receiver, are simple and rapid but may cause signal oscillation phenomena that may lead to errors in determining the flocculation threshold. The company ROFA® markets a probe with an adjustable optical path length (SVA-130® probe) which could avoid such oscillations. However, changing the optical path length requires the intervention of an operator, which considerably increases the measurement time.

The methods currently proposed for measuring the flocculation threshold of asphaltenes in hydrocarbon products therefore have a certain number of drawbacks. They do not necessarily offer the simplicity, speed and precision required for the results, in particular for continuous control of a processing unit, for example visbreaking and/or an efficient mixing unit. Nor do they allow the direct analysis of a wide range of products according to their asphaltene content. They use techniques that may not be easily automated and/or are not very easy to use.

The present invention aims to remedy one or more of the drawbacks mentioned above.

### SUMMARY OF THE INVENTION

The invention relates to a device for measuring the flocculation threshold of a colloidal medium by the addition of an aliphatic solvent, comprising:
at least one measuring cell operating by direct optical transmission and having a measuring chamber defined by fixed walls intended to receive the medium within the measuring chamber, and, associated with each measuring cell:
   a light emitter emitting a light beam entering the measuring chamber along an emission direction,
   a light receiver directly receiving the light beam exiting from the measuring chamber,
   a motorised displacement member of a component selected from the transmitter, the measuring cell and an optical element located between the transmitter and the measuring cell, in a parallel direction to the emission direction,
a management system for the motorised displacement member of each measuring cell arranged to adjust the volume of each measuring chamber through which the light beam passes.

The management system thus makes it possible to modify the volume of the measuring chamber through which the light beam passes, in other words the optical path travelled, and this for a single measuring cell associated with a transmitter and a receiver, the volume of the measuring chamber itself remains fixed. Thus, only one probe is necessary to measure more or less dark products, which avoids the oscillations observed with the device described in document EP1751518 B1. The management system in particular may adjust the illuminated volume of a measuring chamber between a minimum value corresponding to a predefined fraction of the volume of the measuring chamber and a maximum value corresponding to the entire volume of the measuring chamber or to a fraction of the volume of the measuring chamber greater than the previously mentioned fraction.

In addition, the measuring cell operates by direct optical transmission, in other words the light beam emitted by the transmitter is received directly by the receiver, without any intermediate optical reflection device.

The use of a motorised displacement member and management system makes it possible to automate the operation of the device and thus dispense with an operator.

Advantageously, the management system may be arranged to modulate the luminous intensity of the light beam emitted by the transmitter, in other words the amount of light emitted in the emission direction. More precisely, “luminous intensity”, means energy intensity, that is to say a radiometric quantity which is a measure of power (or energy flow) of electromagnetic radiation emitted by a quasi-point source, per unit solid angle, in a given direction. Its unit in the international system is the watt per steradian (W sr⁻¹). In particular, the modulation of the luminous intensity mentioned above is the variation in energy intensity (the power radiated in the emission cone) obtained by varying the electric current through the emitter. When the emitter is a light emitting diode, the energy intensity is almost proportional to the electric current flowing through the diode.

In addition, the measuring chamber of each measuring cell having fixed dimensions, these may be chosen so that the light beam passes through a predetermined amount of material, for example greater than that of the existing probes, thus improving the accuracy of the device even when the volume through which the light beam passes is at a minimum value.

It should be noted that the management system may be connected to other components of the device and arranged to command/control them, such as the transmitter (to control the luminous intensity of the light beam emitted), the receiver (to record the signals emitted), temperature sensors, temperature control member for the medium, or solenoid valves, or even fluid circulation devices, in order to control the distribution of fluids and possibly their circulation, particularly when the device comprises a circuit as described below.

Advantageously, each measuring chamber may have two fixed optical elements forming opposite walls, the transmitter and the associated detector being located outside the measuring chamber, in particular each facing an optical element in the emission direction.

By optical element, is meant a component through which a light beam may pass, particularly without absorbing it.

In some representations, a convergence optical element may be provided between the measuring chamber and the detector in order to converge the light beam exiting the measuring chamber onto the detector. This optical convergence element is therefore located outside the measuring chamber.

In particular, each of the optical elements may be selected from a parallel-sided plate, a spherical lens and an aspherical lens. Preferably, a plate with parallel sides and an aspherical lens may be chosen.

In particular, when the measuring chamber has two plates with parallel sides, an optical convergence element may advantageously be arranged between the measuring chamber of the measuring cell and the receiver in order to converge the light beam exiting the chamber onto the receiver. This optical convergence element comprises for example a lens.

According to one embodiment, each motorised displacement member moves the associated transmitter, the latter emitting the light beam directly onto the measuring chamber.

Advantageously, the measuring device may comprise at least one temperature sensor and at least one temperature control member connected to the management system and the management system may be arranged to control the temperature of the medium.

Advantageously, each measuring cell may comprise a fluid inlet and outlet connecting the measuring chamber to an associated fluid circuit equipped with a fluid flow member. In other words, each measuring chamber of a measuring cell then forms part of a fluid circuit specific to the measuring device according to the invention, which is not in communication with other fluid circuit(s).

Such a fluid system may be formed by one or more lines connected to each other.

In particular, each fluid circuit may comprise one or more of the following:
at least one tank and at least one liquid injection line connected to each tank, optionally connected to the circuit by a valve, in particular a solenoid valve,
a mixing chamber with an inlet and an outlet connected to the fluid circuit,
at least one temperature control member.

This temperature control member may be chosen from a heat exchanger, a heating resistor, a Peltier effect device or other.

Advantageously, the fluid circuit may form a closed loop within which the medium circulates.

The measuring device according to the invention allows the measurements to be carried out in a very short time, making continuous measurements possible. This measurement time may be in the millisecond range, for example 0.5 ms.

Also, advantageously, the device may comprise means for continuously injecting liquid, in particular at a constant flow, into the fluid circuit, in particular into the lines of the fluid circuit. This allows continuous injection of the aliphatic solvent into the circuit. Such a continuous injection while the liquid circulates within the circuit makes it possible to obtain rapid homogenization of the mixture. Due to the very short measurement time, a measurement may be made with the measuring cell while the liquid is circulating in the fluid circuit, without ceasing to add the solvent, which is injected at a constant low flow. This homogenisation will be all the faster as the solvent is injected within the lines of the circuit. These injection means may comprise an injection line, a pump and a solenoid valve.

The device according to the invention may have two or three identical measuring cells, each associated with a transmitter, a receiver and a motorised displacement member, each cell being connected to its own fluid circuit. The motorised displacement member of the measuring cell may be controlled by the same management system.

The invention also has as object a method for measuring the flocculation threshold of a colloidal medium by adding an aliphatic solvent using the device according to the invention, comprising the step of determining, with the aid of the measuring cell of said device, the flocculation threshold after the addition of the amount of aliphatic solvent necessary for flocculation.

The invention also provides a method for measuring the flocculation threshold of a colloidal medium by the addition of an aliphatic solvent, in particular paraffinic, comprising the following steps:
(i) the medium is introduced into a measuring chamber defined by fixed walls of a measuring cell operating by direct optical transmission, the measuring cell forming part of a device for measuring the flocculation threshold further comprising, associated with the measuring cell:
   a light emitter emitting a light beam entering the measuring chamber along an emission direction,
   a light receiver directly receiving the light beam exiting from the measuring chamber,
   a motorised displacement member of a component selected from the transmitter, the measuring cell and an optical element located between the transmitter and the measuring cell, in a parallel direction to the emission direction,
      the measuring device further comprising a system for managing the motorised displacement member of the measuring cell arranged to adjust the volume of each measuring chamber through which the light beam passes and optionally to adjust a luminous intensity emitted by the transmitter,
(ii) the volume of the measuring chamber through which the light beam passes, and optionally a luminous intensity of the transmitter, is automatically adjusted by the management system so as to obtain a signal detectable by the receiver,
(iii) the flocculation threshold is determined with the aid of the flocculation measuring device, after the addition of the amount of paraffinic solvent required for flocculation.

This process may be implemented by the device of the invention.

The device for measuring the flocculation threshold according to the invention makes it possible to carry out measurements in a sufficiently short time, to allow measurements to be taken while the aliphatic solvent is being added. Thus, advantageously, during step (iii), the aliphatic solvent may be added continuously and the measurements are carried out using the measuring device while the aliphatic solvent is being added. In particular, the aliphatic solvent may then be injected into a fluid circuit, in particular into fluid lines, the fluid circuit being connected to the measuring chamber of the measuring cell, this fluid circuit being equipped with a fluid flow member.

Advantageously, these continuous measurements may be carried out at a constant flow of aliphatic solvent.

Advantageously, the probes may be probes emitting in the NIR range and the occurrence of flocculation is identified by determining the absorption peak.

Advantageously, the process may be implemented at a predetermined adjustable temperature, for example by means of a temperature control member. This may allow the product to be heated, for example to facilitate its dissolution, prior to the addition of the aliphatic solvent, but to perform the measurement at a predetermined lower temperature.

Advantageously, the process may comprise a step of diluting (i1) the colloidal medium with a predetermined amount of aliphatic solvent prior to step (i).

According to one embodiment, the colloidal medium comprises asphaltenes.

The invention further provides a method of determining the stability of a mixture comprising asphaltenes by implementing the method of measuring the flocculation threshold of a colloidal medium at least twice according to the invention on a medium containing the mixture and a given amount of aromatic solvent, at different dilution rates. The process for measuring the flocculation threshold may be in particular implemented at least twice in succession in the same measuring cell of a measuring device or simultaneously in two or more identical measuring cells of the same measurement device.

According to one embodiment, the aromatic solvent/aliphatic solvent pair (in particular paraffinic) used is the toluene/n-heptane pair.

### BRIEF DESCRIPTION OF THE FIGURES

FIG. 1 is a representation of the aromaticity graph of the solvent as a function of the inverse of the dilution, i.e. the precipitation curve of a black product which, at a given dilution rate of this same black product, associates the minimum aromaticity of the solvent necessary so that the mixture does not precipitate.

FIG. 2 is a schematic representation of a device according to one embodiment of the invention.

FIGS. 3a and 3b are schematic representations of a measuring cell according to one embodiment of the device of the invention, wherein the transmitter occupies different positions.

FIG. 4 is a schematic representation of a measuring cell according to another embodiment of the device.

FIGS. 5a, 5b and 5c represents respectively the S, Sa and So values of the black product BP3 as a function of the number of tests. FIGS. 6a, 6b and 6c represents the S, Sa and So values of the black product BP14 as a function of the number of tests respectively. All these figures have the same legend, shown only in FIG. 5a.

### DETAILED DESCRIPTION OF THE EMBODIMENTS OF THE INVENTION

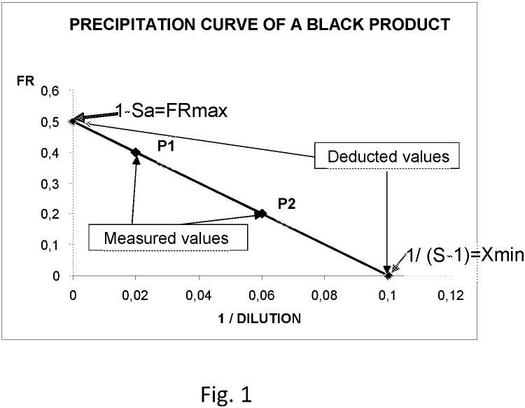

With reference to FIG. 1, the method is described using the device described in document EP1751518 B1 for determining the values of S, So and Sa, for a given black product mixture.

The intrinsic stability of any colloidal system is quantified by a dilution using a paraffinic solvent of a black product, such as a fuel oil, atmospheric (or under vacuum) oil distillation residue, crude oil, previously mixed with an aromatic solvent. This intrinsic stability (S) depends on the aromatic characteristics of the asphaltenes (Sa) and the aromatic characteristics of the medium (So), as described above. The intrinsic stability S of a colloidal system is determined by measuring the flocculation threshold of at least 2 different mixtures. From at least these 2 points, a straight line is drawn, known as the precipitation of a black product (FIG. 1), which allows access to the parameters Sa and S, then by calculation, to the value So.

By adding a paraffinic solvent to the black product, the mixture becomes unstable from a certain dilution rate X min., called “minimum dilution rate”.

The following definitions are used, as defined in the ASTM D7157-18 standard (Revision 2018):
Dilution rate X (ml/g):
   volume of total solvent (aromatic + paraffinic) in millilitres/mass of black product in grams.
intrinsic stability S of the black product:
   S = 1 + Minimum dilution rate. Here we find the notion of S-1 as a stability reserve.

For the experimental measurements, two types of solvents are used, the first is aromatic, consisting essentially of aromatic molecules for the dilution of the sample (for example toluene, xylene, or even 1-methylnaphthalene) and the second is an aliphatic paraffinic solvent (for example n-heptane, cetane, or even iso-octane) to cause flocculation of the asphaltenes.

The flocculation rate FR (“flocculation ratio”) is defined as follows:

FR=volume of aromatic solvent/ total volume of solvent.

The ability of asphaltenes to be peptised (“peptisability of an asphaltene”) is defined by: Sa=1- FRmax, where FRmax is the maximum flocculation rate (at 1/X=0).

The precipitation curve is the function of the flocculation rate FR as a function of the dilution rate, here:

1-Sa=f(1/X)=A + B/X.

A and B are constants that depend only on the sample and allow access to the values of S, So and Sa.

We proceed as follows. We start from a first mixture of a given mass of black product in a given quantity of aromatic solvent and we add in successive increments a paraffinic solvent. The flocculation threshold is determined (in particular by a method using an IR probe) and the dilution rate and the flocculation rate FR associated with the analysed mixture are noted. A first point is obtained, identified by point P1 on the graph (FIG. 1). The operation is repeated, with a starting product that is initially less strongly diluted in the aromatic solvent. This results in another measurement materialised by point P2. With the two points P1 and P2 it is then possible to draw the straight line passing through these points and to obtain limit values (1-Sa) on the y-axis (FRmax or infinite dilution rate) and 1/(S -1) on the x-axis (FR zero). It then becomes possible to access the values of S, Sa then So by calculation.

This technique, which refers to the ASTM D7157-18 standard (Revision 2018), and which consists of the construction of a precipitation curve, from at least two measurement results (three in the standard), to then determine the values of the borderline and null aromatics, is generally followed in the invention. The masses, volumes and products used are conventional in the art of this type of analysis.

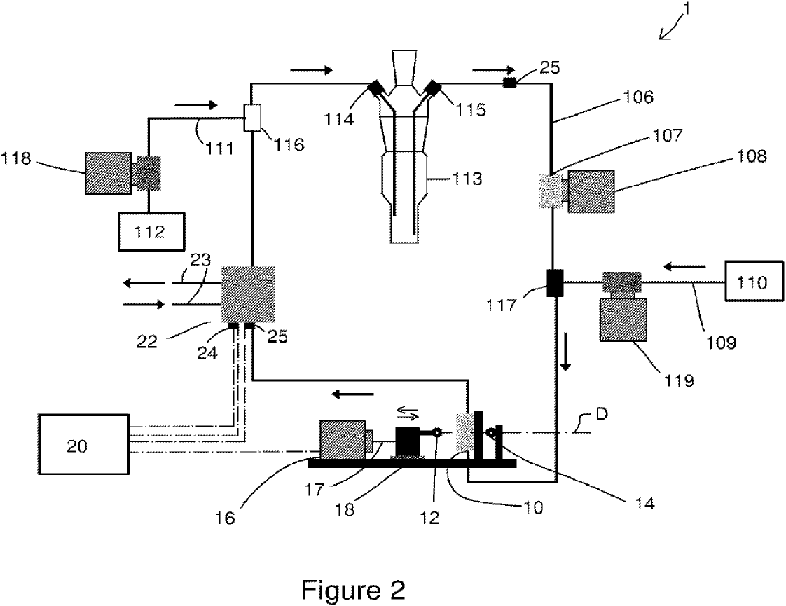

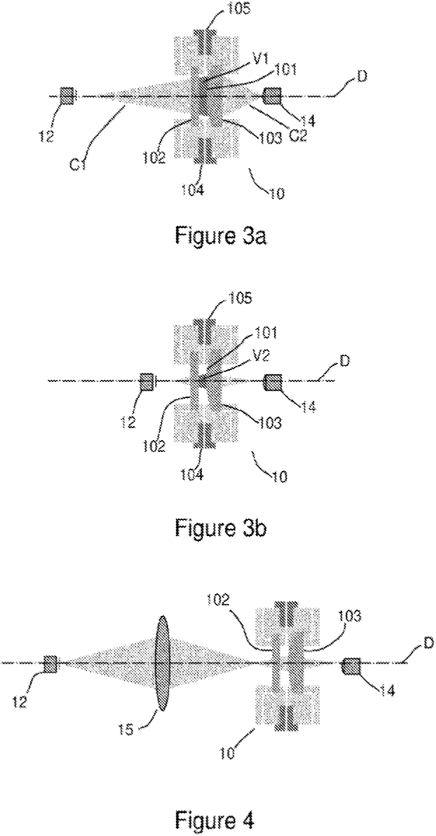

With reference to FIG. 2 and FIGS. 3a, 3b, 4, the device (1) according to the invention comprises a measuring cell (10) operating by direct optical transmission having a measuring chamber (101) of fixed dimensions, defined by fixed walls.

qThe device (1) also comprises, associated with the measuring cell (10), a light emitter (12) emitting (configured to emit) a light beam entering the measuring chamber (101) along an emission direction (D) and a light receiver (14) directly receiving the light beam exiting from the measuring chamber (101). In other words, the light receiver (14) is positioned to directly receive the exiting light beam. In particular it may be positioned in the emission direction (D), on one side of the measuring chamber (101) opposite the side where the light emitter (12) is located, as shown in the figures. The transmitter is a conventional IR emitter, for example a light-emitting diode, and receiver, the latter is a photoelectric receiver able to deliver a current when it receives a luminous flux. The required diameter of the transmitter is 4.0-5.0, for example 4.7-4.8 mm.

The device (1) also comprises a member for motorised displacement member (16) of a component, in this case the transmitter (12), in a parallel direction to the emission direction (D) of the transmitter. The motorised displacement member, configured to move a component, is for example an electric motor, in particular a stepper motor. The shaft (17) of this motor may be connected to the transmitter (12) in order to move it in translation, the transmitter (12) for example is supported on a mobile base (18), for example mounted on rails (not shown).

In the embodiment shown, the device (1) further comprises a management system (20) at least of the motorised displacement member (16) allowing the automation of the volume adjustment of the measuring chamber through which the light beam passes. It is therefore not a question of modifying the volume of the measuring chamber, which is fixed, but only the volume of a portion of the measuring chamber illuminated by the light beam. In the example, the management system is also arranged to control the luminous intensity emitted by the transmitter (12).

This management system (20) may comprise one or more processors of the microprocessor, microcontroller or other type, for example forming part of a computer. In particular the processor(s) comprise(s) of a computer program execution means suitable for implementing the method described in the present invention.

In one embodiment, the management system may be arranged to receive data. The management system may also be arranged to transmit data, particularly to a display device such as a screen. The management system may thus comprise one or more input, output, or input/output interfaces. These may be wireless communication interfaces (Bluetooth, WI-FI or other) or connectors (network port, USB port, serial port, Firewire® port, SCSI port or other).

In one embodiment, the management system may comprise storage means which may be a random access memory or a RAM memory (from the English “Random Access Memory”), an EEPROM (from the English “Electrically-Erasable Programmable Read-Only Memory”), a flash memory, an external memory, or other. These storage means may in particular store the data received, and possibly computer program(s).

In the embodiment shown, unlike the existing conventional probes, the measuring chamber (101) is part of the measuring cell (10) but is not defined by the transmitter (12) and the receiver (14) although that it is located between the latter allowing the light beam to pass through the measuring chamber. The measuring chamber (101) is defined here in part by two fixed optical elements (102, 103) which form opposite walls of the measuring chamber. In other words, the transmitter and receiver are located outside the measuring chamber and are separate from it.

A first optical element (102) located on the side of the emitter (12), in this case a plate with parallel faces, allows the transmission of the light beam from the emitter (12) to the sample located within the measuring chamber (101). A second optical element (103) located on the side of the detector (14), in this case an aspherical lens, makes it possible to focus the light beam transmitted by the sample onto the detector (14).

Other pairs of optical elements previously listed may be considered, however, the configuration shown in the example has the advantage of being particularly efficient. These different optical elements may be made of glass, polymer, metalloid, but also of hybrid material (glass/polymer).

In the example, the transmitter (12) is movable in translation and moved by means of the motorised displacement member (16). The measuring cell (10) and the detector (14) are fixed. The transmitter (12) may be moved between:
a first position (FIG. 3a) wherein the emission cone (C1) of the light beam is at its maximum so that all, or almost all, of the volume (V1) of the measuring chamber (101) is traversed by the light beam, and
a second position (FIG. 3b) wherein the emission cone (C1) of the light beam has a lower apex angle so that only a portion (V2) of the volume of the measuring chamber (101) is traversed by the light beam.

It is thus understood that the movement of the transmitter makes it possible to adjust the volume of the measuring chamber (101) crossed by the light beam.

In both positions, it should be noted that the emission cone of the light beam C2 converges on the fixed detector (14).

Alternatively, the motorised displacement member (16) could move the measuring cell (10), the transmitter and the detector being fixed, or, as shown in FIG. 4, an optical element (15), for example a lens, located between the fixed transmitter (12) and the fixed measuring cell (10). In this case, the half-angle at the apex of the cone of light arriving on the measuring chamber may be varied by moving the lens closer to or further from the measuring cell (10). It is thus understood that the optical element (15) is optional.

In the embodiment shown, the device (1) further comprises two temperature control members (22), in this case for example a heat exchanger within which a coolant liquid (23), may be circulated and a heating member (24). This heating member could also be located around the mixing chamber (113) described below, such as a thermostat block or similar. It also comprises one or more temperature sensors (25), for example a temperature sensor located in the heat exchanger and a temperature sensor in the measuring cell, at the inlet or at the outlet thereof, or in the fluid circuit as shown. The invention is however not limited by a particular position of the temperature sensors. In particular, one may be positioned upstream of the measuring chamber in relation to the fluid flow.

These components may be controlled by the management system (20) which may then be arranged to automatically manage the temperature of the medium.

The measuring cell (10) could be immersed in the medium so that the latter completely fills the measuring chamber. However, preferably, as shown in FIGS. 2 to 4, the measuring cell (10) comprises a fluid inlet (104) and a fluid outlet (105) connecting the measuring chamber (101) to a fluid circuit (106), which is equipped with a fluid flow member (107), in this case a peristaltic pump (107) controlled by a stepping motor (108). The measuring chamber may thus be in the form of a pipe open at both ends, with a closed cross-section.

Specifically, in the example, the fluid circuit (106) comprises:
a first liquid injection line (109) connected to a tank (110) for injecting the first solvent, for example the aromatic solvent,
a second liquid injection line (111) connected to a second tank (112) for injecting the second solvent, for example the paraffinic solvent,
a mixing chamber (113) having an inlet (114) and an outlet (115) connected to the fluid circuit (106), for receiving the medium,
the temperature control member (22) and the heating member (24) as mentioned above.

The injection lines (109) and (111) may be equipped with solenoid valves (116), (117), and a pump (118), (119) which are preferably controlled by the management system (20) for the automation of the device.

The fluid circuit (106) here forms a loop which may therefore be closed for the circulation of the medium within the loop, for example in the direction of circulation symbolised by the arrows in FIG. 2.

It may be possible to provide a heating system for the mixing chamber and a reflux column to enable the product contained in the chamber to be heated under reflux in order to facilitate the dissolution of the sample.

The operation of the device according to the invention is described below.

The sample to be analysed is introduced into the measuring chamber of the measuring cell of the device according to the invention. In the device represented, the sample is introduced into the mixing chamber before being circulated through the circuit and into the measuring chamber. In particular, the product volume is sufficient to completely fill at least the measuring chamber.

In the example, this introduction step is followed by a step of adding the aromatic solvent to the product to form the medium to be analysed. The sample is then diluted by the aromatic solvent before circulating within the measuring chamber of the measuring cell.

This is followed by an adjustment step wherein the volume of the measuring chamber of the measuring cell through which the light beam passes is adjusted, before the addition of paraffinic solvent, that is to say before flocculation. It may further be advantageous to adjust the luminous intensity emitted by the transmitter. This adjustment step allows obtaining a signal that may be detected by the receiver. During this step, it is preferable to first set the luminous intensity emitted by the transmitter and then set the volume in order to obtain a detectable signal. By detectable signal, is meant a signal that may be distinguished from background noise and is not saturated.

Finally, the flocculation threshold is determined with the aid of the measuring device, after the addition of the amount of paraffinic solvent required for flocculation. To this end, the paraffinic solvent is gradually added and the drop in transmission corresponding to the flocculation of the asphaltenes is noted. This determination is done by conventional techniques, for example, by measuring the absorption peak.

The volume adjustment of the measuring chamber through which the light beam passes is carried out automatically, by a computer program predetermined during the construction of the device. This automatic adjustment may comprise an alteration of the luminous intensity emitted by the transmitter.

It may in fact be preferable to modulate the luminous intensity emitted by the transmitter in order to reach a setpoint value corresponding to a minimum value measurable by the detector. This luminous intensity setting may be obtained by varying the intensity of a direct current supplied to the transmitter.

In a known manner, a detector may detect a light beam in a determined detection range, corresponding to a percentage of the light emitted by the transmitter: below the minimum value of this range, no signal is detected, above the maximum value of the range, saturation of the receiver causes a loss of sensitivity. The setpoint value is generally chosen in a part of the detection range close to the minimum value.

The setting for example is made as follows. The luminous intensity emitted by the transmitter is first set to its minimum value, corresponding for example to a current of 6 mA, while the volume of the measuring chamber through which the light beam passes is at a maximum value, for example the order of 500 µl. The luminous intensity emitted by the transmitter is then increased until the setpoint value or a maximum value of the luminous intensity emitted is reached, corresponding for example to a current of 100 mA. As soon as the setpoint value may not be reached by increasing the emitted luminous intensity to this maximum value, the volume of the measuring chamber is gradually reduced until the setpoint value is reached or until a minimum volume value, for example in the order of 10 µl. The measurements will then be carried out under these conditions. In particular, the luminous intensity emitted by the transmitter and the volume of the measuring chamber remain fixed as the paraffinic solvent is diluted. In this way, the signal may be measured with good accuracy with a single, appropriately adjusted measuring cell, which saves considerable time for the operator.

The minimum value of the luminous intensity emitted by the transmitter corresponds for example to a value below which the accuracy of the measurement is too low to distinguish a signal from background noise. This minimum value corresponds for example to a current of 6 mA.

The minimum value for the volume of the measuring chamber corresponds for example to 10 µl. It may be determined experimentally by measurements with highly opaque samples. This minimum volume could be increased in order to allow/improve the detection of flocculation of media containing very low amounts of asphaltenes.

According to an advantageous embodiment, implementing in particular the device described with reference to the figures, the introduction stage comprises a dissolution phase, during which the medium is introduced into the mixing chamber (113), in a sufficient quantity to completely fill the circuit (106), then the temperature control member for the medium to a dissolution temperature by means of the temperature control member (22) and the heating member (24), or by means of a temperature control member surrounding the mixing chamber (113). The medium contained in the mixing chamber (113) may also be kept under agitation. The aromatic solvent is then injected into the circuit and the medium and aromatic solvent are circulated in the circuit (106) by means of the pump (107) for sufficient time to obtain a homogeneous mixture.

This dissolution phase may optionally be followed by a pre-dilution phase with the paraffinic solvent, during which a predetermined quantity of this solvent may be injected into the circuit. This is done in the case of a very aromatic and stable product or when the product is too dark and the luminous intensity emitted by the detector reaches its maximum without having detected the flocculation volume.

A cooling phase is then carried out during which the temperature is regulated to a predetermined test temperature by means of the temperature control member (22).

This is followed by a dosing phase during which the paraffinic solvent is gradually added. This addition of solvent may be achieved by incremental or continuous addition. The detector signal is then acquired and recorded either after each addition of solvent, or during the addition of solvent. In the latter case, the flow of solvent introduction into the circuit may be constant, for example of the order of 1 mL/minute. It should be noted that the product to be analysed circulates in the circuit during the addition of the solvent and the acquisition of the signal. This dosing phase may be stopped by an operator, when the maximum volume of the mixing cell has been reached or when a predetermined number of incremental additions has been made or when a predetermined volume of solvent has been added.

It is then possible to carry out a cleaning phase for example by circulating the aromatic solvent in the circuit.

The invention is described with reference to a device comprising a single measuring cell. It should be noted however that the device of the invention may comprise various identical independent measuring cells, for example three, in order to simultaneously perform three tests in parallel on a product.

In addition, the device according to the invention makes it possible to obtain a possible spectral range of application for the measurements which is very broad. The device according to the invention is suitable for determining the S, Sa and So values for all types of residues and fuels and is practically not limited as to the nature of the medium to be tested. As the device comprises one type of measuring cell, it is possible to carry out various measurements to measure the same product in a shorter time. In this way 3 measurements may be made and thus 3 points on the curve may be obtained and thereby a good repeatability of the measurements for S, Sa and So. Finally, the determination method according to the invention may be implemented at an ambient temperature or at a predetermined temperature, which makes it possible to measure the parameters S, Sa and So at a given temperature and to check their evolution as a function of temperature, since the stability of asphaltenes is temperature dependent.

In general, the aromatic solvent/paraffinic solvent pair used in the invention is the toluene/n-heptane pair.

### EXAMPLES

The following examples illustrate the invention without limiting it.

### Example 1

Measurements were carried out on 13 different black products samples for which the S, Sa values were measured and So calculated, on the one hand with a method using the SVA-130® probes proposed by the company ROFA implementing the method described in the ASTM D7157-18 standard (Revision 2018) (“Measurement Method A” in Table 1 below) and on the other hand with the device and the method in accordance with the present invention (“Measurement Method B”).

The device according to the present invention is of the type described with reference to FIGS. 2 and 3. The measuring cell in particular comprises a parallel-sided plate and an aspherical lens, the distance between the window and the lens being 0.5 mm at the centre and 3 mm at the edges.

The volume of the circuit loop here is 4 ml. The measurements are made while the fluid is circulating at a speed of approximately 10 mL/min. The test temperature here is room temperature. It is possible to heat the aromatic solvent/product mixture to accelerate the dissolution of the latter, particularly in the case of vacuum residues. Heating from 60° C. to 100° C. is sufficient to dissolve the product in this case in a few minutes. In some cases (very stable products), a pre-dilution with n-heptane was carried out before the start of the measurements in order to avoid a saturation of the detector.

In this example, the black products labelled BP1 to BP13 correspond to:
BP1: Visco-reduced atmospheric residue prefluxed low sulphur content (pre-diluted with fluxant);
BP2: Visco-reduced atmospheric residue prefluxed high sulphur content;
BP3: Visco-reduced atmospheric residue prefluxed high sulphur content;
BP4: Visco-reduced vacuum residue;
BP5, BP7, BP8: Vacuum residue;
BP6: Low sulphur content vacuum residue;
BP9, BP11: slurry;
BP10, BP12, BP13: very unstable fuel mixtures.

It should be noted that the values obtained with Measurement Method B according to the invention are close to the values obtained with Measurement Method A, the SVA-130® probes allows implementation of the ASTM D7157-18 standard (Revision 2018) in compliance with the repeatability and reproducibility conditions defined in this standard.

For each of the 13 measurements, the correlation coefficient R² of the precipitation curve (flocculation rate FR as a function of the inverse of dilution) constructed with 3 points (P1, P2 and P3) varies from 0.9817 to 0.9999, which is greater than the minimum R² value (0.98) required by the standard.

Furthermore, the automation of the analysis allows the complete analysis to be carried out in less than one hour with Measurement Method B according to the invention, whereas it takes more than two hours for Method A in particular because of the operator time required to modify the optical path of the SVA-130® probes. In addition, Measurement Method B according to the invention is also faster than using a device and method in accordance with document EP1751518 B1 due in particular to the automation of the dilution.

TABLE 1

Comparison of the results of the S and Sa measurements, and the calculation of So, with the ROFA SVA-130® probes (Measurement Method A) and the device and method in accordance with the invention (Measurement Method B)

| Products | Measurement Method A | | | Measurement Method B | | |
|---|---|---|---|---|---|---|
| | S | Sa | So | S | Sa | So |
| BP1 | 1.62 | 0.51 | 0.8 | 1.89 | 0.483 | 0.975 |
| BP2 | 1.73 | 0.45 | 0.93 | 1.93 | 0.432 | 1.1 |
| BP3 | 1.93 | 0.62 | 0.74 | 2.01 | 0.612 | 0.779 |
| BP4 | 1.64 | 0.36 | 1.04 | 1.65 | 0.353 | 1.07 |
| BP5 | 7.9 | 0.85 | 1.16 | 7.59 | 0.883 | 0.89 |
| BP6 | 4.27 | 0.86 | 0.6 | 5.01 | 0.86 | 0.701 |
| BP7 | 4.29 | 0.81 | 0.83 | 4.92 | 0.815 | 0.923 |
| BP8 | 1.53 | 0.46 | 0.83 | 1.67 | 0.43 | 0.95 |
| BP9 | 1.38 | 0.14 | 1.18 | 1.43 | 0.172 | 0.19 |
| BP10 | 1.31 | 0.61 | 0.51 | 1.35 | 0.659 | 0.461 |
| BP11 | 1.63 | 0.24 | 1.25 | 1.76 | 0.258 | 1.3 |
| BP12 | 1.69 | 0.6 | 0.68 | 1.73 | 0.615 | 0.666 |
| BP13 | 1.43 | 0.44 | 0.81 | 1.45 | 0.408 | 0.856 |

### Example 2

In order to compare the repeatability values obtained on the measurements of S, Sa and then the calculated So, accessible by the method using the ROFA SVA-130® probes (Measurement Method A) and the automated method whose device and process are the subject of the present invention (Measurement Method B), 2 samples BP3 and BP14 were selected, sample BP3 is defined in Example 1, sample BP14 is crude from Kuwait, liquid at a temperature below 30° C.

Tables 2 and 3 below show the average values calculated for 11 separate measurements for Measurement Method B according to the invention and average values calculated over ten separate measurements for Measurement Method A. The repeatability and reproducibility values are calculated using the formulae in the ASTM D7157-18 standard (Revision 2018) from the average calculated for the measurements of each of the Measurement Methods A and B are also included in these Tables. Tables 2 and 3 also show the standard deviation for the 11 measurements of Measurement Method B, as well as the repeatability calculated according to the general formula: 2 x square root of 2 x standard deviation, or 2.83 x standard deviation.

The efficiency shown in these Tables is the ASTM repeatability report (according to ASTM D7157-18 -Revision 2018) on the repeatability calculated with the general formula.

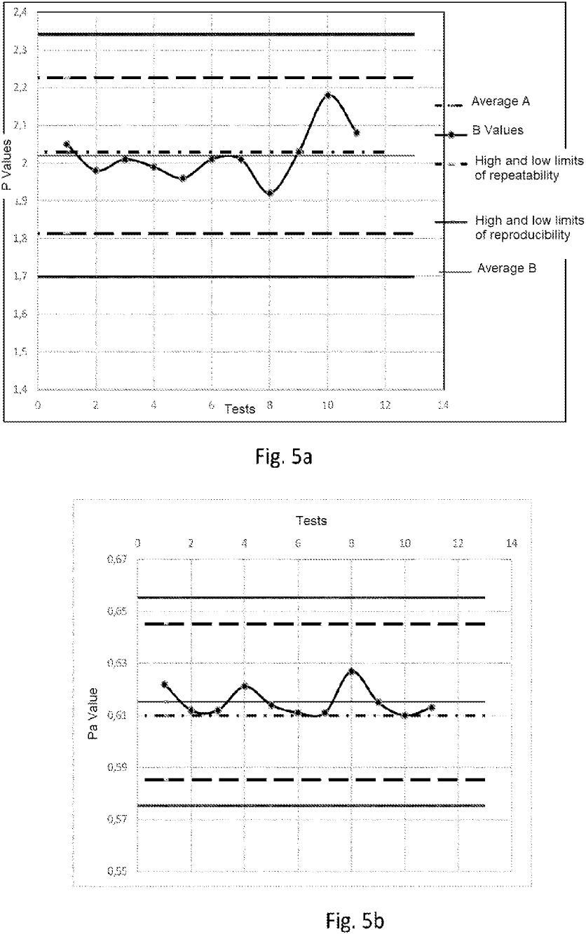

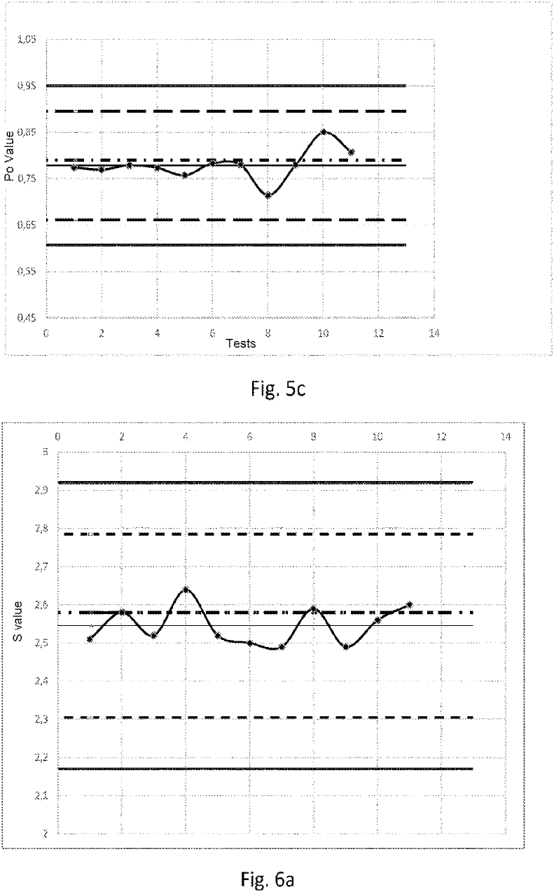

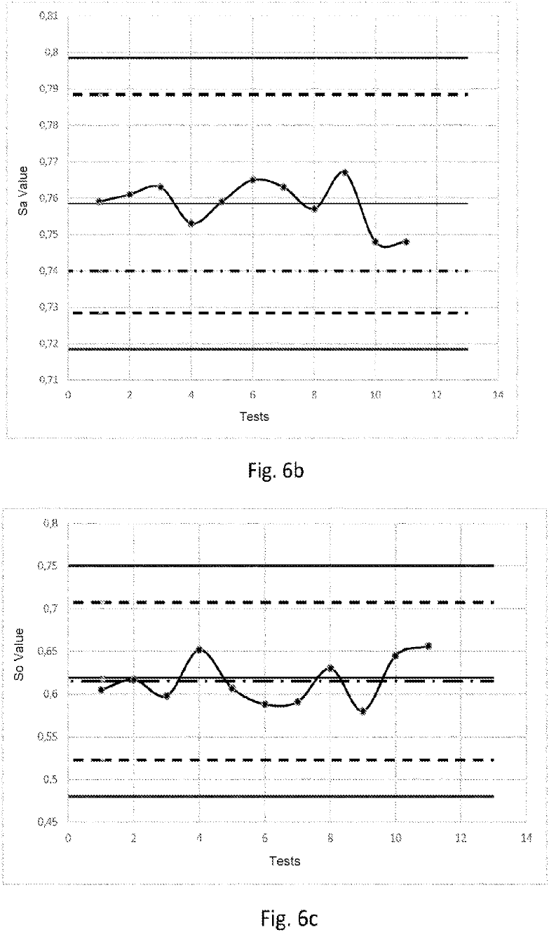

FIGS. 5a, 5b and 5c represent the S, Sa and So values of the black product BP3 respectively, FIGS. 6a, 6b and 6c represents the S, Sa and So values of the black product BP14 for the 11 measurements.

In each of these figures, are represented:
high and low limits of S, Sa and So taking repeatability into account (high and low repeatability limits), calculated by adding and subtracting the repeatability value calculated for Measurement Method B from the average of the measurements calculated for Measurement Method B respectively,
high and low limits S, Sa and So taking reproducibility into account (high and low reproducibility limits), calculated by adding and subtracting the reproducibility value calculated for Measurement Method B from the average of the measurements calculated for Measurement Method B respectively,
the average of the values obtained with the Measurement Method B according to the invention (Average B),
the values obtained with the Measurement Method B according to the invention (B values),
the average of the values obtained with Measurement Method A (Average A).

It is thus noted that the values appearing in Tables 2 and 3 are relatively close between Measurement Methods A and B, also shown by curves 5a, 5b, 5c relating to sample BP3 and curves 6a, 6b, 6c relating to sample BP14.

Furthermore, the notion of efficiency expressed in Tables 2 and 3 makes it possible to compare whether the repeatability of the ASTM D7157-18 standard (2018 Revision) is lower or greater than the repeatability of the device according to the invention. In particular, the lower the repeatability, the more the values are repeatable and therefore less variable. It should be noted in particular that the efficiency value is always greater than 1, which means that the repeatability of the device according to the invention is lower than the repeatability of the ASTM D7157-18 standard (Revision 2018).

In each FIGS. 5a, 5b, 5c, 6a, 6b, 6c, it may be seen that the minima and the maxima of the curves of the tests performed according to Measurement Method B are between the low and high repeatability and reproducibility limits. In other words, the differences in values between various measurements obtained with the Measurement Method B according to the invention are low for both types of products.

TABLE 2

Repeatability measurements of the S, Sa and So values on sample BP3 obtained with the ROFA SVA-130® probes (Measurement Method A) and the device and method in accordance with the invention (Measurement Method B)
BP3

| | Measurement Method A | | | Measurement Method B | | |
|---|---|---|---|---|---|---|
| | S | Sa | So | S | Sa | So |
| Mean | 1.930 | 0.620 | 0.740 | 2.020 | 0.615 | 0.779 |
| ASTM reproducibility | 0.313 | 0.040 | 0.163 | 0.322 | 0.040 | 0.171 |
| ASTM repeatability | 0.200 | 0.030 | 0.111 | 0.206 | 0.030 | 0.117 |
| Standard deviation | - | - | - | 0.07 | 0.01 | 0.03 |
| Repeatability | - | - | - | 0.193 | 0.016 | 0.093 |
| Efficiency | - | - | - | 1.07 | 1.90 | 1.26 |

TABLE 3

Repeatability measurements of the S, Sa and So values on sample BP14 obtained with the ROFA SVA-130® probes (Measurement Method A) and the device and method in accordance with the invention (Measurement Method B)
BP14

| | Measurement Method A | | | Measurement Method B | | |
|---|---|---|---|---|---|---|
| | S | Sa | So | S | Sa | So |
| Mean | 2.580 | 0.740 | 0.660 | 2.548 | 0.757 | 0.619 |
| ASTM reproducibility | 0.378 | 0.040 | 0.145 | 0.375 | 0.040 | 0.136 |
| ASTM repeatability | 0.242 | 0.030 | 0.099 | 0.240 | 0.030 | 0.093 |
| Standard deviation | - | - | - | 0.05 | 0.006 | 0.027 |
| Repeatability | - | - | - | 0.14 | 0.018 | 0.076 |
| Efficiency | - | - | - | 1.66 | 1.65 | 1.22 |

### Example 3

The device according to the invention has also been tested with products containing less than 0.5% mass of asphaltenes:
BP15: Arabian crude extra light containing 0.45% mass of asphaltenes
BP16: Olmelca crude containing 0.3% mass of asphaltenes.

Table 4 shows the S, Sa and So values obtained. For each of the measurements, the correlation coefficient R² of the precipitation curve is greater than 0.98. This example demonstrates that the device according to the invention makes it possible to determine the flocculation threshold of black products even at very low asphaltene contents.

TABLE 4

Determination by Measurement Method B of the S, Sa and So values for crude PB15 and BP16
Measurement Method B

| | S | Sa | So | R² |
|---|---|---|---|---|
| BP15 | 2.07 | 0.779 | 0.457 | 0.9820 |
| BP16 | 2.33 | 0.802 | 0.46 | 0.9954 |

1. Device for measuring the flocculation threshold of a colloidal medium by adding an aliphatic solvent, comprising:
at least one measuring cell operating by direct optical transmission and having a measuring chamber defined by fixed walls, intended to receive the medium within the measuring chamber, and, associated with each measuring cell:
a light emitter configured to emit a light beam entering the measuring chamber along an emission direction,
a light receiver receiving the light beam directly exiting from the measuring chamber,
optionally an optical element located between the transmitter and the measuring cell,
a motorised displacement member of a component selected from the transmitter, the measuring cell and the optical element, in a parallel direction to the emission direction,
a management system of the motorised displacement member of each measuring cell arranged to adjust the volume of each measuring chamber through which the light beam passes.

2. Measuring device according to claim 1, characterised in that each measuring chamber has two fixed optical elements forming opposite walls, the associated emitter and detector being located outside the measuring chamber, optionally each measuring chamber is defined by two optical elements, each selected from a parallel-sided plate, a spherical lens and an aspherical lens.

3. Measuring device according to claim 1, characterised in that the management system is arranged to modulate the light intensity of the light beam emitted by the transmitter.

4. Measuring device according to claim 1a, characterised in that each motorised displacement member moves the associated transmitter, the latter being configured to emit the light beam directly onto the measuring chamber.

5. Measuring device according to claim 1, characterised in that it comprises at least one temperature sensor and at least one temperature control member connected to the management system and in that the management system is arranged to control the temperature of the medium.

6. Measuring device according to claim 1, characterised in that each measuring cell comprises a fluid inlet and outlet and in that the measuring device comprises a fluid circuit associated with each measuring chamber and connected to the fluid outlet thereof, the fluid circuit being equipped with a fluid flow member.

7. Measuring device according to claim 6, characterised in that each fluid circuit comprises one or more of the following:
at least one tank and at least one liquid injection line connected to each tank,
a mixing enclosure having an inlet and an outlet connected to the fluid circuit,
at least one temperature control member.

8. Measuring device according to claim 6, characterised in that the fluid circuit forms a closed loop within which the medium circulates.

9. Measuring device according to claim 6, characterised in that it comprises means for continuously injecting liquid, and in particular at a constant flow into the fluid circuit.

10. A process for measuring the flocculation threshold of a colloidal medium by adding an aliphatic solvent comprising the following steps:
(i) the medium is introduced into a measuring chamber defined by fixed walls of a measuring cell operating by direct optical transmission, the measuring cell forming part of a device for measuring the flocculation threshold according to claim 1,
(i 1) optionally, a step of diluting said medium with a predetermined quantity of aliphatic solvent prior to step (i),
(ii) the volume of the measuring chamber through which the light beam passes, and optionally a luminous intensity emitted by the transmitter, is automatically adjusted using the management system so as to obtain a signal detectable by the receiver,
(iii) the flocculation threshold is determined using the flocculation measuring device after the addition of the amount of aliphatic solvent required for the flocculation, optionally, the aliphatic solvent is added continuously, in particular at a constant flow, and the measurements are carried out using the measuring device while the aliphatic solvent is being added.

11. Method according to claim 10, wherein the emitter emits a light beam in the NIR range and the occurrence of flocculation is identified by determining the absorption peak.

12. Method according to claim 10, wherein the occurrence of flocculation is determined at a predetermined adjustable temperature.

13. Method according to claim 10, wherein the medium comprises asphaltenes.

14. Method according to claim 10, wherein the light consists of wavelengths belonging to a spectral range selected from among the near infrared spectral range and the infrared spectral range.

15. Method for determining the stability of a mixture comprising asphaltenes by successively implementing the process according to claim 10 at least twice on a medium containing the mixture and a given quantity of aromatic solvent, at different dilution rates, optionally, the aromatic solvent/aliphatic solvent pair used is the toluene/n-heptane pair.
